(19) United States
(12) Patent Application Publication
(10) Pub. No.: US 2021/0267689 A1
(43) Pub. Date: Sep. 2, 2021

(54) COMPARISON OF VARUS AND VALGUS INFORMATION

(71) Applicant: Strive Orthopedics, Inc., Green Bay, WI (US)

(72) Inventor: Padraic Obma, Green Bay, WI (US)

(73) Assignee: Strive Orthopedics, Inc., Green Bay, WI (US)

(21) Appl. No.: 17/261,150

(22) PCT Filed: Jul. 18, 2019
(86) PCT No.: PCT/US19/42340

§ 371 Date: Jan. 18, 2021

Related U.S. Application Data
(60) Provisional application No. 62/699,961, filed on Jul. 18, 2018.

(51) Int. Cl. A61B 34/10; A61B 5/107; A61B 5/11; A61B 5/00; A61B 34/30

(52) U.S. Cl. CPC A61B 34/10; A61B 5/1071; A61B 5/1128; A61B 5/0002; A61B 34/30; A61B 5/4585; A61B 5/0077; A61B 5/4523

(57) ABSTRACT

Sensors may be attached to a patient's skin near a joint. An image of the joint and sensors may be captured and a bone centerline may be determined based on the image. A first set of measurements, which may include varus and valgus information, may be captured at the sensors, and may be captured while the patient is in a first position. The first set of measurements may then be transmitted to a handheld device. A second set of measurements corresponding to a second positon of the patient may be captured at the sensors. The second set of measurements may also include varus and valgus information and may also be transmitted to the handheld device. A difference between the first set of measurements and the second set of measurements, including the difference between the varus and valgus measurements, may be determined.

### PRIORITY CLAIM

This application claims priority to U.S. Provisional Patent App. No. 62/699,961, filed Jul. 18, 2018.

### BACKGROUND OF THE INVENTION

Ligament balancing is an important part of knee replacement surgery. X-rays and CT scans are used to perform robotic-assisted joint surgery, such that a surgeon relies almost exclusively on bone anatomy to perform the surgery. However, this reliance on bone anatomy fails to account for the ligaments, tendons, and muscles that also make up the knee joint and surrounding area. The most important soft tissues to assess and consider are the Medial Collateral Ligament (MCL), Lateral Collateral Ligament (LCL), Anterior Cruciate Ligament (ACL), Posterior Cruciate Ligament (PCL), the hamstring tendons, the quadriceps, and the calf muscles.

If a patient has a loose MCL or other ligament or tendon before undergoing knee replacement surgery, the patient can end up with a knee that is either too loose or too tight if the surgeon performs the surgery based only on bone alignment. This results in the need for additional diagnostics and treatment, extending healing time for the patient. However, a diagnostic platform that uses both bone anatomy and soft tissue (ligaments, tendons, muscle) anatomy to quantify and balance the knee joint can allow for a successful, balanced knee replacement.

### BRIEF DESCRIPTION OF THE DRAWINGS

FIG. 1 is an example of the sensors consistent with the present disclosure.

FIG. 2 is an example of the sensors as applied consistent with the present disclosure.

FIG. 3 is a side view of the example of the sensors as applied consistent with the present disclosure.

FIG. 4 is an example method consistent with the present disclosure.

### DETAILED DESCRIPTION

One diagnostic method that may be used may include diagnostic sensors that a patient is able to use in his or her home. When coupled with advanced imaging technology, such as X-rays, CT scans, and/or Magnetic Resonance Imaging (MRI), the use of diagnostic sensors may allow a surgeon to monitor changes in, for example, joint alignment, joint characteristics, and degree of stress on ligaments, tendons, and/or muscles. For example, the use of sensors may be used to monitor degrees of Varus, or bowed leg, or valgus, or knock-knee, conditions, to assist in providing a more complete representation of a patient's knee joint.

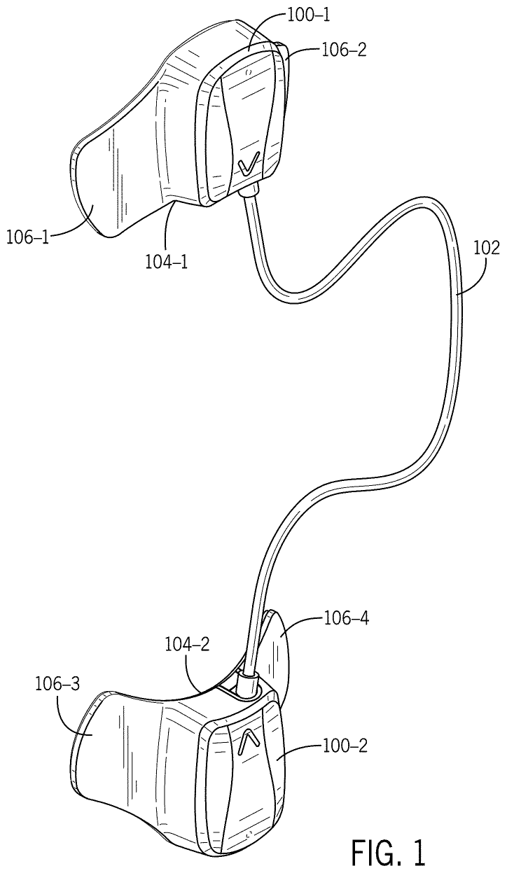

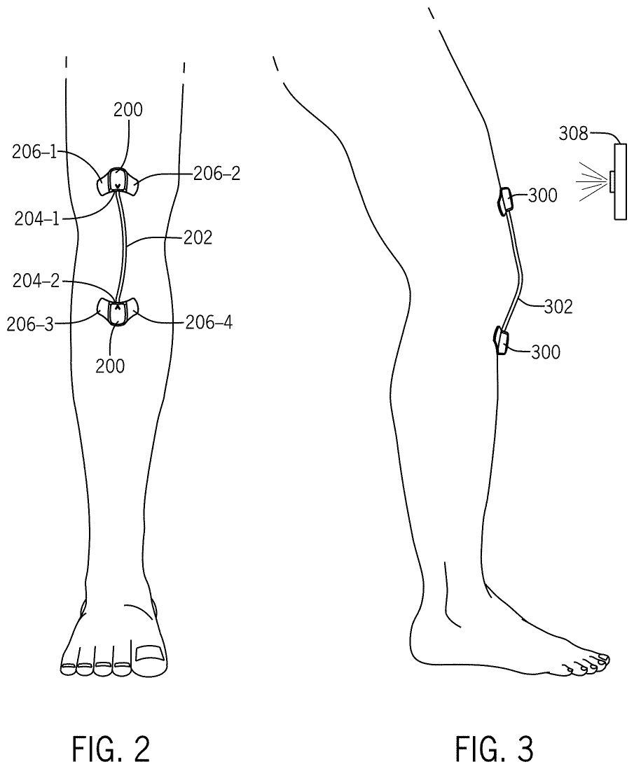

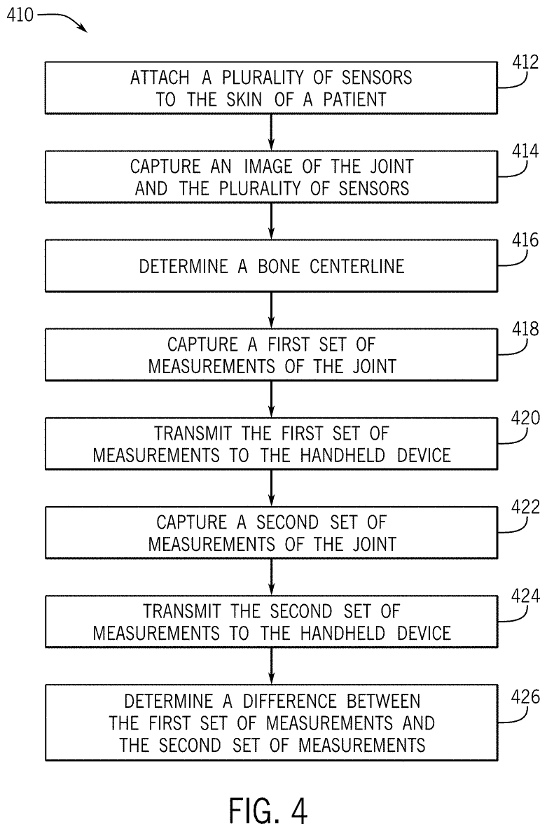

The diagnostic method (see 410 of FIG. 4) may include a pair of sensors 100-1, 100-2, shown in FIG. 1, that may be coupled to one another, either via a wired connection 102 or with a wireless connection (not shown). Each sensor may have a portion that forms an inverted shallow U shape 104-1, 104-2 (opening 104), with a pair of opposing outwardly and downwardly-extending wings 106-1, 106-2, 106-3, 106-4 (collectively, wings 106). Each sensor may be adapted to be placed on the skin adjacent to a joint, with the opening between the wings positioned closest to the joint. For example, when applied to a knee, as in FIG. 2, the opening 204 between the wings 206 of each sensor 200 is placed next to the patella. The sensors may correspond to the sensors disclosed in U.S. patent application Ser. No. 15/916,043, by this inventor, the contents of which are hereby incorporated in their entirety by reference.

The sensors may have a known size and color, which may serve as a reference for measurements taken while wearing the sensors. A patient may apply the sensors (see 412 of FIG. 4) and, using a handheld device, such as handheld device 308 shown in FIG. 3, capture an image of the sensors and the joint. The image may be used to determine a bone centerline of the joint. In some examples, the image may be transmitted to the handheld device 308, and a bone centerline may be determined using the image including the joint and the sensors, with the known characteristic of the sensors being used as a reference for the bone centerline. See 414 of FIG. 4. In some examples, a bone centerline may be determined on each side of the joint; that is, a plurality of bone centerlines may be determined. These bone centerlines may then be averaged or otherwise combined to provide a bone centerline measurement having increased accuracy.

Each sensor may include a variety of measurement instruments. These may include a gyrometer, for measuring limb and joint rotation, an accelerometer, to measure speed and directional changes in limbs and joints, and a magnetometer, to provide a fixed spatial point between the two sensors such that the directional orientation of the patient is able to be measured. Examples are not limited to these particular measurement instruments, however, and any other measurement instruments may be used.

Once a patient has applied the sensors around a joint, the patient may be instructed to perform a variety of movements while wearing the sensors. By performing the movements with the sensors on, the sensors may capture information using the measurement instruments. See FIG. 4. For example, a patient may be instructed to sit, then stand, to capture information pertaining to the degree of stress or laxity in ligaments, such as the MCL or LCL. When the patient is seated, or in a first position, the sensors may obtain a first set of measurements of the joint. See 418. The measurements may include stress on the joint, which may correspond to a degree of varus or valgus condition experienced by the patient, as well as joint alignment. At 420, the first set of measurements captured at 418 may be transmitted to the handheld device.

The patent may then be instructed to stand, or move to a second position, becoming weight bearing, and the sensors may obtain a second set of measurements of the joint, at 422. Again, the measurements may include of the stress on the joint, corresponding to a degree of varus or valgus condition experienced by the patient, and joint alignment. The second set of measurements captured at 422 may be transmitted to the handheld device at 424.

In some examples, the sensors may determine that the degree of varus/valgus experienced by the patient is different when the patient is standing versus when the patient is seated or lying down. See 426 of FIG. 4. This may correspond to a loss of cartilage on the joint and/or a loose or tight ligament. This difference information may be combined with patient demographic information (e.g., height and weight) and with the bone centerline determined by the image capture using the sensor to determine how loose or tight the MCL, LCL, or any other ligament or tendon is. By determining the laxity or tightness of the ligament or tendon, a surgeon is able to adjust bone cuts prior to performing surgery to perform a more accurate and personalized surgery.

In some examples, the ligament data captured using the sensors may be used to instruct the surgeon to perform soft tissue releases (loosening or partially cutting certain ligaments to make them looser). This allows the surgeon to perform surgery and get the desired results while not being forced to adjust bone cuts. At present, whether or not to perform soft tissue releases is done entirely at the surgeon's discretion, with minimal data to support or suggest a preferred course of action. However, by having a patient perform additional diagnostics using the sensors and combining the information received from the diagnostics with additional diagnostics (X-ray, MRI, and similar), the surgeon may be able to preserve the ACL, MCL, and/or LCL when appropriate, providing a more customized implant process and experience.

In some examples, the ACL and/or PCL is preserved. The use of the sensors to perform diagnostics regarding varus and valgus conditions may assist a surgeon in assessing the stability of the ligaments. For instance, assume an average PCL translates 3.0 millimeters (mm) posterior; however, a patient performing the diagnostics with the sensors has a posterior PCL translation measurement of only 1.0 mm. This indicates that the PCL is tighter than average and, during the knee replacement surgery, the surgeon has the information necessary to adjust the bone cuts by, for example, adding additional posterior slope or removing the PCL, to prevent the patient from developing a stiff knee.

Moreover, the sensors may be used in combination with video capture to assess joint translation, motion, and rotation while a patient is moving the joint normally. For example, the sensors may be used with video capture to see a patient walking. The gait, joint translation, and knee motion may be used to determine the stability of ligaments, such as the ACL, in order to provide additional recommendations regarding how the soft tissue should be handled during the surgery.

In some examples, the measurements captured by the sensors may be transmitted to a robotic assistance device, such as a robotic surgical device. The robotic surgical device may receive the first set of measurements and the second set of measurements, as well as the bone centerline information, and may perform the comparison between the first set of measurements and the second set of measurements. Again, by determining a difference in a patient's measurements in different positions, for example, between sitting and standing, the robotic assistance device may be able to perform at a more refined level by, for example, adjusting the surgical cuts being performed.

In some examples, joint data may be collected over a prolonged period of time, rather than relatively soon before a joint replacement is needed. For example, the joint can be analyzed as part of a routine physical examination. This may provide data corresponding to the joint's condition prior to the development of a problem or issue with the joint, and may allow the sensors to have a more refined set of information regarding the normal condition of the joint. In turn, this more refined set of information may permit an improved joint replacement or implant design when the patient does require an implant, such as a 3D printed joint to more accurately mirror the patient's original anatomy.

In addition, when the sensors are used over an extended period of time to examine a patient's joint, a surgeon is able to account for the precise ways in which the patient's joint has changed over time. This may aid the surgeon in customizing the surgery to restore the patient's function to as close as possible to the original, pre-surgery state.

1. A method, comprising:
attaching a plurality of sensors to the skin of a patient near a joint, wherein:
each sensor of the plurality of sensors has a known characteristic;
a first sensor of the plurality of sensors is placed on a first side of the joint;
a second sensor of the plurality of sensors is placed on a second side of the joint, wherein the second side of the joint is opposite the first side of the joint; and
the plurality of sensors is movable with respect to the joint;
capturing, by a handheld device, an image of the joint and the plurality of sensors;
determining a bone centerline, wherein:
the bone centerline is determined based on the captured image of the joint and the plurality of sensors; and
a bone centerline is determined on each side of the joint;
capturing, at the plurality of sensors, a first set of measurements of the joint, wherein:
the first set of measurements further comprises:
varus information;
valgus information; and
joint alignment; and
the first set of measurements is captured while the patient is in a first position;
transmitting the first set of measurements to the handheld device;
capturing, at the plurality of sensors, a second set of measurements of the joint, wherein:
the second set of measurements further comprises:
varus information;
valgus information; and
joint alignment; and
the second set of measurements is captured while the patient is in a second position;
transmitting the second set of measurements to the handheld device; and
determining a difference between the first set of measurements and the second set of measurements.

2. The method of claim 1, further comprising:
determining, based on the determined difference between the first set of measurements and the second set of measurements, an amount of stability in at least one tendon of the joint;
adjusting, based on the determined amount of stability in at least one tendon, a surgical plan for replacement of the joint.

3. The method of claim 1, further comprising transmitting the captured first set of measurements and second set of measurements and the determined bone centerline to a robotic assistance device, wherein the transmitted measurements and bone centerline aid in operation of the robotic device.

4. The method of claim 1, wherein:
each sensor of the plurality of sensors has a portion that forms a shallow inverted U shape with a pair of opposing outwardly and downwardly-extending wings; and
attaching a sensor of the plurality of sensors to the skin of a patient further comprises placing the sensor such that the opening between the wings is positioned closest to and facing the joint.
